(19) United States
(12) Patent Application Publication
(10) Pub. No.: US 2021/0307632 A1
(43) Pub. Date: Oct. 7, 2021

(54) NONINVASIVE BLOOD-PRESSURE MEASURING DEVICE

(71) Applicant: PULSION MEDICAL SYSTEMS SE, Feldkirchen (DE)

(72) Inventors: Torsten SCHEUERMANN, München (DE); Aaron WEBER, Markt Schwaben (DE); André HEIN, Esslingen am Neckar (DE); Thomas THALMEIER, Dorfen (DE)

(21) Appl. No.: 17/272,227

(22) PCT Filed: Aug. 27, 2019
(86) PCT No.: PCT/EP2019/072847

§ 371 Date: Feb. 26, 2021

(30) Foreign Application Priority Data
Aug. 29, 2018 (DE) 10 2018 006 845.6

(51) Int. Cl. A61B 5/022; A61B 5/0225

(52) U.S. Cl. CPC A61B 5/02241; A61B 2562/08; A61B 5/02255

(57) ABSTRACT

The invention relates to a measuring device for continuously determining the intra-arterial blood pressure in a finger of a hand, the measuring device comprises a base part and a cuff part. A light source for near-infrared light and a photodetector are provided for the finger. The light sources and the photodetectors are connected to an associated optical emission surface or optical collector surface via a respective so-called light pipe for coupling emitted light into the finger tissue or decoupling non-absorbed light from the finger tissue. The cuff-side and base-part-side sections of the light pipes are connected to one another via separable optical contact points at the interface between the cuff part and the base part. On the base-part side, a cover glass closes flush with the housing of the base part and is attached to the contact points.

### TECHNICAL AREA

The present invention relates to a non-invasive blood pressure measuring device, in particular a measuring device for continuously determining the intra-arterial blood pressure on at least one finger of a hand.

### PRIOR ART

The (in particular arterial) blood pressure of a patient is one of the most important measured variables in medical technology, and the known associated, especially non-invasive, measurement technology is extremely diverse. This applies above all to measurement technology for continuous monitoring of blood pressure over a longer period of time, for example in intensive care medicine, but also in emergency medicine and during surgical interventions.

For reasons of good accessibility, the blood pressure measuring device is often attached to the patient's limbs, for example an applanation tonometric sensor in the radial artery on the forearm or a finger sensor operated photoplethysmographically based on the so-called Vascular Unloading Technique according to Peňáz. Such pressure measuring devices are known, for example, from U.S. Pat. Nos. 4,406,289, 4,524,777, 4,726,382, WO 2010/050798 A1, WO 2000/059369 A1, WO 2011/045138 A1, WO 2011/051819 A1, WO 2011/051822 A1, WO 2012/032413 A1, and WO 2017/143366 A1.

In the Vascular Unloading Technique, near-infrared light is radiated into a finger, and the pulsatile (pulse-shaped) blood flow (actually the changing blood volume) in the finger is determined on the basis of the non-absorbed portion captured by a photodetector. For this process, also known as photoplethysmography (PPG), the (near-infrared) light is usually generated with the aid of one or more light-emitting diodes (LED), which work with one or more wavelengths, and detected with the aid of one or more light-sensitive receiver diodes (photodiodes). Instead of diodes, other types of photoreceivers are basically also suitable.

A control system then keeps the plethysmographically recorded flow (or the detected blood volume) and thus the resulting photoplethysmographic signal (volume signal v(t)) constant by applying a counterpressure in a cuff (cuff pressure) pc(t) on the finger. This counterpressure pc(t) is usually regulated by a fast valve or valve system in conjunction with a pump. The related control of the valve or the valve system is carried out by a control unit, which is preferably implemented with a microcomputer. The main input signals are the PPG signal v(t) and the cuff pressure pc(t). The pressure pc(t) required to keep the PPG signal v(t) constant then corresponds to the intra-arterial blood pressure pa(t).

To this end, it must be possible to change the cuff pressure pc(t) at least as quickly as the intra-arterial blood pressure pa(t) changes, so that the real-time condition is fulfilled. The upper limit frequency of pa(t) and thus the highest rate of pressure change is greater than at least 20 Hz, which is definitely a challenge for a pressure-control system. The result of this is that the pressure control by means of a valve or valve system is advantageously located in the immediate vicinity of the cuff. If the air lines are too long, there is a risk of losing this upper limit frequency condition due to the low-pass effect of the lines.

A mechanical valve is known from U.S. Pat. No. 4,406,289 which regulates the counterpressure in the finger cuff with the desired accuracy when it is supplied with a linear pump. The valve is housed in a housing on the distal forearm and thus supplies the finger cuff with the pressure pc(t) via a short tube.

U.S. Pat. No. 4,524,777 describes a pressure-generation system for the vascular unloading technique, a constant cuff pressure Pc also being generated with a linear pump, which is superimposed with pressure fluctuations Δpc(t) from a shaker or a driving actuator connected in parallel.

U.S. Pat. No. 4,726,382 discloses a finger cuff for the Vascular Unloading Technique which has hose connections for the supply of the cuff pressure pc(t). The length of the air tubes extends to the pressure-generation system, which in turn is attached to the distal forearm.

WO 2000/059369 A1 also describes a pressure-generation system for the Vascular Unloading Technique. The valve system here consists of a separate inlet and a separate outlet valve. While a relatively linear proportional pump must be used in U.S. Pat. Nos. 4,406,289 and 4,524,777, this system allows the use of simple, inexpensive pumps, since disruptive harmonics can be eliminated by the arrangement of the valves. Furthermore, the energy consumption of the simple pump can be significantly reduced by the valve principle.

WO 2004/086963 A1 discloses a system for the Vascular Unloading Technique in which the blood pressure can be continuously determined in one finger, while the measurement quality is checked in the neighboring finger (watchdog function). After a while, the system automatically replaces the measuring finger with the monitoring finger.

WO 2005/037097 A1 describes a control system for the Vascular Unloading Technique with several interlinked control loops.

WO 2010/050798 A1 discloses a pressure-generation system (front end) attached to the distal forearm with only one valve, to which a finger cuff can be attached for the Vascular Unloading Technique.

With the pressure-generation system described in WO 2011/045138 A1 for the Vascular Unloading Technique, the energy consumption of the pump is reduced similar to WO 2000/059369 and harmonics can be eliminated.

WO 2011/051819 A1 discloses an implementation of the Vascular Unloading Technique, improved by means of digital electronics, for increased stability and further miniaturization.

WO 2011/051822 A1 describes a method for the Vascular Unloading Technique, in which the measured signals v(t) and pc(t) are processed to increase long-term stability and to determine further hemodynamic parameters. In particular, a method for eliminating effects resulting from vasomotor changes in the finger arteries and a method for determining cardiac output (CO) are disclosed.

WO 2012/032413 A1 describes novel finger sensors that have a disposable part for single use. The cuff that comes into contact with the finger is housed in the disposable part for reasons of hygiene, whereas the associated pressure-generation and pressure-control system is housed in a reusable part. Accordingly, a separable pneumatic connection must be provided in this case between the disposable part and the reusable part.

As a rule, the pressure-generation and pressure-control system in the prior art is attached to the distal forearm, proximal to the wrist, which has significant disadvantages: This point is often used for intravenous access; also, the intra-arterial access at the distal end of the radial artery should be free for emergencies. Such accesses can be blocked by the pressure-generation and pressure-control system and its attachment. In addition, the system can slip or tilt during operation. This can have a detrimental effect on the fit of the sensors. The fit of the sensors would also be improved if the finger to be measured or the corresponding hand is in a certain resting position.

To overcome this problem, publication WO 2017/143366 A1 proposes a measuring system for the continuous determination of the intra-arterial blood pressure on at least one finger of a hand, with at least one finger sensor, with a plethysmographic system, with at least one light source, preferably LED, with one or more wavelengths, and at least one light sensor, and at least one inflatable cuff, as well as with a pressure-generation system with at least one valve regulated in real time with the aid of the plethysmographic system for generating a pressure in the cuff which essentially corresponds to the intra-arterial blood pressure in the finger, with the measuring system having a housing with a surface that serves as a support surface for the at least one finger and the adjacent areas of the palm. The hand rests here on a support under which there are essential components that were attached to the forearm in conventional systems.

Similar to previously mentioned WO 2012/032413 A1, the cuff is housed in a disposable part that can be separated from the housing (and thus from the hand support). Accordingly, a separable pneumatic connection must be provided in this case between the disposable part and the reusable part.

In the known systems, the light-emitting diodes and photodiodes for emitting and detecting the near-infrared measuring radiation, possibly embedded in transparent silicone, are arranged directly on the finger. When the light-emitting diodes and photodiodes are arranged in a reusable part, there is the problem that the exposed light-emitting elements must be subjected to cleaning and disinfection before they can be reused. The need for an easy-to-clean design restricts the degree of freedom in the design. Otherwise, the need to accommodate the light-emitting diodes and photodiodes in the immediate vicinity of the finger represents a limitation of the geometric configuration of the device. When the light-emitting diodes and photodiodes are arranged in a disposable part, on the other hand, there is the problem that electrical connections must be provided between the disposable part and the reusable base unit, and that the costs for production of the disposable part increase. The input of heat when the electrical components have contact with skin is also perceived as negative.

### PRESENTATION OF THE INVENTION

In light of the restrictions that exist in conventional systems, the object of the present invention is to improve measuring devices of the type mentioned at the beginning with respect to production and use.

According to one aspect of the present invention, this object is achieved with a device according to claim 1.

Preferred embodiments of the invention can be implemented according to any of the dependent claims.

The present invention thus in particular provides a measuring device for the continuous determination of the intra-arterial blood pressure on at least one finger of a hand, which has a base part and a cuff part which can be connected to the base part without tools and can be separated from the base part without tools, preferably designed as a disposable item, and also a radiation source for emitting light into the finger through an optical emission surface, a photodetector for detecting a portion of the light captured by an optical collector surface and not absorbed in the finger, a cuff for receiving the finger arranged in the cuff part which can be filled with a fluid (usually a gas, for example air, although implementations with a liquid as a fluid are also advantageously possible), and a pressure-control system arranged at least partially in the base part for controlling a fluid pressure in the cuff as a function of the detected unabsorbed portion of the light. In this case, the radiation source and/or the photodetector is arranged in the base part, with a respective non-fiber-optic light guide connection (so-called light pipe) being provided between the radiation source arranged in the base part and/or the photodetector and the optical emission surface or the optical collector surface, said light guide connection being at least partially arranged in the cuff part, and the respective light guide connection being separable from the base part together with the cuff part and having an optical contact point for coupling light from the base part into the cuff part or decoupling light from the cuff part into the base part.

In the present application, light is understood according to the usual definition to be electromagnetic radiation in the infrared, visible, and ultraviolet range. For the usual photoplethysmographic application, this is usually near-infrared light (about 700 to 1100 nm wavelength). In principle, light of different wavelengths can be used, in particular for the integration of additional functions such as measuring oxygen saturation, detection of fluorescent dyes, etc.

The light pipes can be manufactured from different glass materials such as quartz glass or also from suitable transparent plastics such as PMMA and polycarbonate, in particular they can be cast and possibly ground, in which one skilled in the art can choose the material according to the conditions in the individual case (optical quality, power of the radiation source or sensitivity the photodetector, material costs, biocompatibility, resistance to aging, especially resistance to yellowing, wear resistance, etc.). For production, one skilled in the art can utilize the range of suitable machining processes, for example ultra-precision machining, glass grinding, etc., depending on the material.

By providing suitable reflection surfaces within the geometry of the light pipes, one skilled in the art can optimize the beam path towards a directional, loss-optimized light transmission. The light pipe geometry can advantageously be adapted individually for different sizes of the cuff part. One skilled in the art gains degrees of freedom in the design, which enables, for example, the use of cuff parts with different dimensions for children's hands and adult hands with one and the same base part. Angle of incidence and beam profiles (divergent/convergent) are adaptable according to the anatomy. The exact arrangement of the light source and photodetector are then no longer dictated by the anatomy. The cuff part can thus be of different sizes, with it being possible for the distance between the light guides in the base part or the distance between the light source and the photodetector in the base part to remain constant.

By virtue of the fact that the radiation source and/or the photodetector are arranged in the (reusable) base part, the production costs for the cuff part, which is preferably designed as a disposable item, can be kept low. Correspondingly, the costs of use per patient can be reduced when using disposable cuff parts.

By avoiding electrical components near or directly on the skin, biocompatibility may be improved. The heat input to tissue can be significantly reduced.

Preferably, the radiation source and the photodetector are arranged in the base part, and a respective non-fiber-optic light guide connection is provided, which is at least partially arranged in the cuff part, both between the radiation source arranged in the base part and the optical emission surface and between the photodetector and the optical emission surface.

The device can thus advantageously be implemented in such a way that there is no electrical line connection between the base part and the cuff part. However, the cuff part can have an electronic component for wireless identification of the cuff part, for example an RFID tag, so that an associated query element in the base part can ensure that only suitable cuff parts are used during operation. Likewise, a component for identifying the cuff part can advantageously serve to prevent the reuse of a cuff part designed as a disposable component.

Dispensing with electrical contacts between the base part and the cuff part can increase both patient safety and functional reliability.

Alternatively, the cuff part may advantageously have an electronic component for identification of the cuff part, and an interface for querying the electronic component, as a single electrical line connection between the base part and cuff part.

According to a preferred embodiment, the radiation source and the photodetector can be arranged on a common circuit board. A driver switch for the radiation source can also be particularly advantageous on the board and/or an amplifier circuit can be arranged for the photodetector. Due to the typically low currents in the μA range, short line lengths are particularly advantageous between the photodiode (photodetector) and the amplifier circuit, which, in addition to cost-effective production and compact design, also speaks in favor of equipping a common circuit board with the corresponding electronic components.

A least one lens may advantageously be provided or a lens geometry can be integrated at the transition between the radiation source and the associated light guide connection and/or between the photodetector and the associated light guide connection.

The optical contact point for coupling light from the base part into the cuff part and/or the optical contact point for decoupling near-infrared light from the cuff part into the base part can advantageously also be provided with at least one lens, or a lens geometry can be integrated into the light guide at the transition.

According to an advantageous refinement, the optical contact point for coupling light from the base part into the cuff part and/or the optical contact point for decoupling light from the cuff part into the base part is provided with at least one cover glass.

According to a further advantageous refinement, the optical emission surface and/or the optical collector surface is equipped with a Fresnel structure for directed coupling in and out of the measuring radiation.

The arrangement according to the invention with light guides offers the possibility of taking further technical measures to improve or adapt the optical transmission path, in particular the coating of reflective surfaces of the light guides, for example by vapor deposition or sputtering of metals such as silver or gold in particular. Further advantageously
Diffraction gratings can be provided in the light guides to influence the light path,
The optical transmission of the interfaces, in particular contact surfaces (such as the emission surface, collector surface, contact point), can be improved by anti-reflective coatings, for example made of silicon dioxide,
Bandpass filters can be introduced into the beam path by coating the coupling surface or collector surface,
Light guides can be coated with a material that is impermeable in the decisive wavelength range and is suitable for preventing crosstalk.

In order to prevent crosstalk between the radiation source and the photodetector, infrared blockers, for example, can be placed between the two elements. Radiation blockers in the housing can also prevent radiation from the environment from reaching the detector. This is an advantage of the installation position of the photodetector inside the base part.

In principle, every variant of the invention described or indicated in the context of the present application can be particularly advantageous, depending on the economic, technical, and possibly medical conditions in the individual case. Unless otherwise stated, or as far as technically feasible in principle, individual features of the described embodiments can be exchanged or combined with one another and with features known per se from the prior art.

In particular, the technology used to build up pressure in the cuff and to regulate the pressure can in principle be designed as known from the prior art.

The invention is explained in more detail below by way of example with reference to the accompanying schematic drawings. The drawings are not to scale; in particular, for reasons of clarity, the relationships between the individual dimensions do not necessarily correspond to the dimensional relationships in actual technical implementations. Corresponding elements are identified by the same reference numerals in the individual figures.

### BRIEF DESCRIPTION OF THE FIGURES

FIG. 1 shows schematically a device according to the invention with a patient's hand placed thereon in a side view.

FIG. 2 shows the same device as in FIG. 1, but without the hand and in a front view, i.e. from the left in FIG. 1.

FIG. 3 shows an enlarged view of FIG. 2 with schematically sketched photoplethysmographic components.

FIG. 4a shows the device as shown in FIG. 1 but without the hand and with marking of the sectional plane for the representation from FIG. 4b.

FIG. 4b is a sectional view of a cutout of the device as shown in sectional plane A-A′ from FIG. 4, wherein break line B-B′ of the cutout is also indicated in FIG. 3.

FIG. 5 shows the base part and cuff part separated from one another in a side view, similar to FIGS. 1 and 4a.

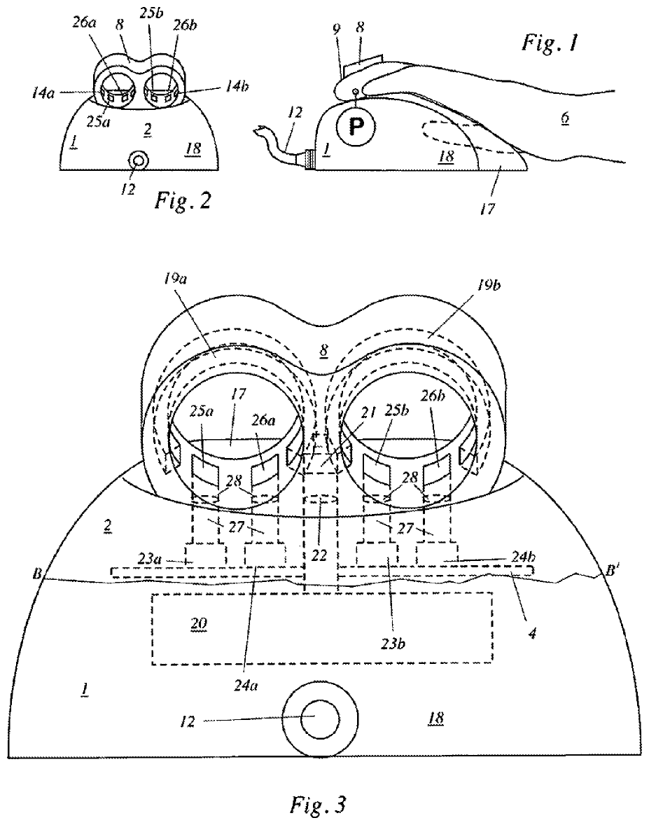

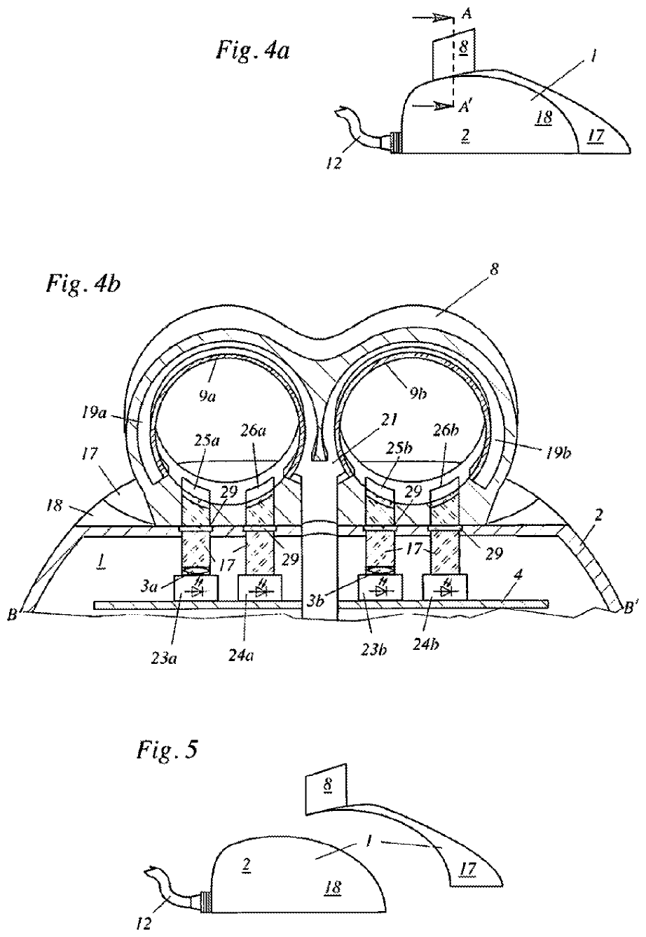

The blood pressure measuring device 1 is designed as a photoplethysmographic measuring system that functions according to the Vascular Unloading Technique. Measurement components, that is to say in particular electronic components 23a, 23b, 24a, 24b, and mechanical components of the pressure-generation and pressure-control system 20 can in principle be implemented similarly to the prior art mentioned at the beginning. Essential components of the exemplary embodiment described are sketched in FIG. 2 and especially FIGS. 3 and 4b, which show the blood pressure measuring device 1 shown in a side view in FIGS. 1 and 4a in the front view (from the left in FIGS. 1 and 4a) or sectional view (FIG. 4b). Elements arranged within the housing 2 of the base part or within the cuff part are indicated by dashed lines in FIG. 3.

The cuff part 8 is designed to accommodate two fingers, which makes it possible to measure alternately on both fingers. For reasons of hygiene, the cuff part 8, together with the palm rest 17, is designed as a disposable item, which is attached to the reusable base part 18 in a detachable manner by means of a plug-in connection. FIG. 5 shows the

The two inflatable finger cuffs 19a, 19b are connected to the pressure-generation and pressure-control system 20 via a distributor 21 and a connection 22 at the interface between the cuff part 8 and the base part 18. In this case, the connection 22 is preferably equipped with a valve (not shown) that closes the connection on the base-part side flush with the housing 2 of the base part 18 when the base part 18 and cuff part 8 are not connected to one another. In alternative embodiments, the finger cuffs 19a, 19b can also be connected separately to a (optionally also respective) pressure-generation and pressure-control system 20 and can thus be controlled separately.

For each of the two fingers, a light source 23a, 23b for near-infrared light, for example a light-emitting diode, and a photodetector 24a, 24b are provided, which are arranged on a common circuit board 4 which also supports the driver switches (not shown) for the light sources 23a, 23b and the amplifier circuits (not shown) for the photodetectors 24a, 24b.

The light sources 23a, 23b and the photodetectors 24a, 24b are connected to an associated optical emission surface 25a, 25b or optical collector surface 26a, 26b for coupling emitted light into the finger tissue or decoupling unabsorbed light from the finger tissue via a respective light pipe 27, i.e. a light guide not designed as a fiber bundle. The optical emission and collector surfaces 25a, 25b, 26a, 26b are equipped with a Fresnel structure for the directional coupling in and out of the measuring radiation.

The light emitted by the respective light source 23a, 23b is coupled into the respective light pipe 27 via the respective lens 3a, 3b.

The cuff-side and base-part-side sections of the light pipes 27 are connected to one another via separable optical contact points 28 at the interface between the cuff part 8 and the base part 18. On the base-part side, a cover glass 29, for example mineral glass or sapphire glass, which closes flush with the housing 2 of the base part 18 and is as scratch-resistant as possible, is attached to the contact points.

The pressure-generation and pressure-control system 20 regulates the cuff pressure in accordance with the signal received by one of the photodetectors 24a, 24b so that the portion of the near-infrared light emitted by the associated light source 23a, 23b that is not absorbed in the corresponding finger remains as constant as possible, i.e. a counterpressure which varies according to the pulsatile portion of the arterial blood pressure is generated and transferred to the respective finger via the flexible cuff membranes 9a, 9b, so that the blood volume area present in the respective finger area (and plethysmographically detected by the respective light source-detector pair 23a, 24a or 23b, 24b) remains approximately constant. The counterpressure in the cuffs 19a, 19b regulated accordingly by the pressure-generation and pressure-control system 20 is detected as a blood pressure measurement signal by a sensor in the pressure-generation and pressure-control system 20 and can be output to a patient monitor via a suitable electronic interface through the cable 12.

The device 1 is also supplied with power via the cable 12.

1. A measuring device for continuous determination of an intra-arterial blood pressure on a finger of a hand, the measuring device comprising:
a base part;
a cuff part that is configured to connect to, and separate from, the base part without tools;
a radiation source configured for emitting light into the finger through an optical emission surface;
a photodetector configured for detecting a portion of the light captured by an optical collector surface and not absorbed in the finger;
a cuff, which is arranged in the cuff part and can be filled with a fluid, for receiving the finger;
a pressure control system arranged at least partially in the base part for regulating a fluid pressure in the cuff as a function of the detected non-absorbed portion of the light, wherein at least one of the radiation source or the photodetector is arranged in the base part; and
a respective non-fiber-optic light guide connection that is at least partially arranged in the cuff part between at least one of the radiation source arranged in the base part or the photodetector and at least one of the optical emission surface or the optical collector surface,
wherein the respective non-fiber-optic light guide connection has an optical contact point that is separable from the base part together with the cuff part for at least one of coupling light from the base part into the cuff part or decoupling light from the cuff part into the base part.

2. The measuring device according to claim 1, wherein the radiation source and the photodetector are arranged in the base part, and
the respective non-fiber-optic light guide connection is provided both between the radiation source arranged in the base part and the optical emission surface and between the photodetector and the optical emission surface.

3. The measuring device according to claim 2, wherein there is no electrical line connection between the base part and the cuff part.

4. The measuring device according to claim 3, wherein the cuff part has an electronic component for wireless identification of the cuff part.

5. The measuring device according to claim 2, wherein the cuff part has an electronic component for identification of the cuff part, and an interface for querying the electronic component, as a single electrical line connection between the base part and the cuff part.

6. The measuring device according to claim 2, wherein the radiation source and the photodetector are arranged on a common circuit board.

7. The measuring device according to claim 6, wherein a driver switch for at least one of the radiation source or an amplifier circuit for the photodetector is further arranged on the circuit board.

8. The measuring device according to claim 1, wherein at least one lens or a lens geometry integrated into the respective non-fiber-optic light guide connection is provided at least one of at a transition between the radiation source and the respective non-fiber-optic light guide connection or at a transition between the photodetector and the respective non-fiber-optic light guide connection.

9. The measuring device according to claim 1, wherein the optical contact point is provided with at least one lens or a lens geometry integrated into the respective non-fiber-optic light guide connection.

10. The measuring device according to claim 1, wherein the optical contact point is provided with at least one cover glass.

11. The measuring device according to claim 1, wherein at least one of the optical emission surface or the optical collector surface is equipped with a Fresnel structure.
